(19) United States
(12) Patent Application Publication
(10) Pub. No.: US 2006/0127976 A1
(43) Pub. Date: Jun. 15, 2006

(54) Use of oxalate deficient aspergillus niger strains for producing a polypeptide

(76) Inventors: Thibaut Jose Wenzel, Leiden (NL); Jean-Marc Maurice Claude Ladriere, Vendegies-Sur-Ecaillon (FR); Rogier Meulenberg, Delft (NL)

Correspondence Address: NIXON & VANDERHYE, PC, 901 NORTH GLEBE ROAD, 11TH FLOOR, ARLINGTON, VA, 22203, US

(21) Appl. No.: 10/544,207

(22) PCT Filed: Feb. 5, 2004
(86) PCT No.: PCT/EP04/01173

§ 371 Date: Aug. 17, 2005

(30) Foreign Application Priority Data
Feb. 5, 2003 (EP) 03100236.3

(51) Int. Cl. C12P 21/06; C12N 9/32; C12N 1/16; C12N 15/74

(52) U.S. Cl. 435/69.1; 435/204; 435/254.3; 435/484

(57) ABSTRACT

The invention relates to oxalate deficient A. niger strains for the production of a given enzyme, wherein the oxalate deficient strain produces at least the same amount of enzyme as the wild type strain it originates from under the same culture conditions. Preferably, the oxalate deficient A. niger strain produces more enzyme than the wild type strain it originates from under the same culture conditions. More preferably, the oxalate deficient A. niger strain is such that when the strain has been transformed with an expression construct comprising a gene coding for an enzyme, said strain produces at least the amount of the enzyme the wild type strain it originates from would produce under the same culture conditions, when the wild type strain has also been transformed with the same expression construct as the oxalate deficient strain. The invention also relates to method for obtaining such oxalate deficient A. niger strain. The present invention further relates to method for producing an enzyme, wherein an oxalate deficient A. niger strain that produces at least the same amount of enzyme as the wild type strain it originates from under the same culture conditions is used.

### FIELD OF THE INVENTION

The invention relates to oxalate deficient Aspergillus niger strains for producing a polypeptide, to their use and to a method for obtaining such strains.

### BACKGROUND OF THE INVENTION

Oxalic acid is an undesirable by-product that accumulates in the culture supernatant of cells during fermentation and causes difficulties in the downstream processing of the desirable compound.

Four Russian prior art documents, named Ru1-Ru4 (defined hereafter), describe how to obtain oxalate deficient Aspergillus niger (A. niger) strains using classical mutagenesis methods. Oxalate deficient A. niger strains are defined as strains that produce less oxalic acid than the parental strain they originate from. They demonstrate that the choice of the mutagen agent is not critical: UV, or chemicals, or a combination of both as mutagen agents would lead to the obtention of oxalate deficient A. niger strains.

They use a chromatography assay to select strains that produce less oxalic acid or more citric acid than the parental strain they originate from. They do not envisage to use these strains for producing polypeptides.

Ru1: On methods of selecting A. niger mutants with altered capacity to synthesize organic acids, ID Kasatkina and E. G. Zheltova, Mikrobiologiya, vol 34, no 3, p 511-518, May-June 1965.

Ru2: RU2089615, New strains of A. niger has properties of producer of citric acid and can be used in microbiological industry (DW1998-249164).

Ru3: The variability of A. niger, a producer of citric acid, under the influence of the separate and combined action of nitrosomethylurea and ultraviolet rays, E. Y. Shcherbakova, Z. S. Karadzhova and V. P. Ermakova, Mikrobiologiya, vol 43, no 3, p 508-513, May-June 1974.

Ru4: Change in the ratio of citric acid and oxalic acids in A. niger under the influence of mutagenic factors, V. M. Golubtsova, E. Y. Shcherbakova, L. Y. Runkovskaya and V. P. Eramkova, Mikrobiologiya, vol 48, no 6, p 1060-1065, November-December 1979.

Another publication, WO 00/50576 describes that oxaloacetate hydrolase deficient host cells can be used for producing desirable compounds, such as polypeptides, primary and secondary metabolites. These host cells have less oxaloacetate hydrolase activity than the parental cells they originate from. As a result, these oxaloacetate hydrolase (OAH) deficient cells produce less oxalic acid than the parental cells they originate from. This patent application does not show experimental data demonstrating that an oxaloacetate hydrolase deficient cell is a suitable polypeptide producer. Furthermore, Pedersen et al, (Pedersen, H., et al, Metabolic Eng., (2000) 2, 34-41) later described that oxaloacetate hydrolase deficient Aspergillus niger strains transformed with a DNA construct comprising the DNA sequence encoding the glucoamylase enzyme are not able to produce the glucoamylase enzyme at the level the wild type strain they originate from does under the same culture conditions: the mutants produce 50% less glucoamylase than the wild type. Such a mutant is not suited as a polypeptide producer in an industrial setting.

There is still a need for oxalate deficient A. niger strains that are able to produce at least the amount of a polypeptide a wild type strain would produce and that can be used as polypeptide producer in an industrial setting.

### DETAILED DESCRIPTION OF THE INVENTION

Oxalate deficient A. niger strains suitable for the production of a given polypeptide or enzyme in an industrial setting have been isolated, wherein surprisingly the oxalate deficient strain produce at least the same amount of polypeptide or enzyme as the wild type strain they originate from under the same culture conditions. Preferably, the mutants produce at least the amount of polypeptide or enzyme the A. niger strain CBS 513.88 produces under the same culture condition.

In this application, A. niger strain CBS 513.88 is taken as a reference of wild type oxalate levels obtainable in an A. niger culture, as a reference of wild type polypeptide level obtainable in an A. niger culture and as a reference of intracellular OAH activity obtainable in an A. niger culture. Oxalate deficient A. niger strains are defined as strains that produce less oxalate than the A. niger strain CBS 513.88 under the same culture conditions. Preferably, the oxalate deficient A. niger strains used produce no more than half the amount of oxalate that the wild type strain they originate from produces under the same culture conditions. More preferably, the oxalate deficient A. niger strains used produce no more than one third of the amount of oxalate that the wild type strain they originate from produces under the same culture conditions. Most preferably, the oxalate deficient A. niger strains used produce no more than one fifth of the amount of oxalate that the wild type strain they originate from produces under the same culture conditions. More preferably, the oxalate deficient A. niger strains used produce no more than half the amount of oxalate that the A. niger strain CBS 513.88 produces under the same culture conditions. More preferably, the oxalate deficient A. niger strains used produce no more than one third of the amount of oxalate that the A. niger strain CBS 513.88 produces under the same culture conditions. Most preferably, the oxalate deficient A. niger strains used produce no more than one fifth of the amount of oxalate that the A. niger strain CBS 513.88 produces under the same culture conditions. According to a preferred embodiment of the invention, the oxalate deficient A. niger strain used has been obtained by applying the method defined later in this application.

Preferably, the oxalate deficient A. niger strains of the invention are strains that produce more of a given polypeptide than the wild type strain they originate from under the same culture conditions. More preferably, the oxalate deficient A. niger strain produces more of a given polypeptide than the A. niger CBS 513.88 under the same culture conditions.

A large variety of systems for detection of polypeptide are known to the skilled person. Detection systems include any possible assay for detection of polypeptide or enzymatic activity. By way of example these assay systems include but are not limited to assays based on colorimetric, photometric, turbidimetric, viscosimetric, immunological, biological, chromatographic, and other available assays.

Preferably, if the polypeptide produced is an enzyme, the amount of active enzyme produced is determined by measurement of its activity in a model reaction (see examples).

Preferably, the oxalate deficient A. niger strains of the invention are strains having a detectable intracellular OAH activity as detected in a model reaction (see experimental information in the Examples) More preferably, the oxalate deficient A. niger strains of the invention are strains having an intracellular OAH activity, which is ranged between 0.1 and 100% of the intracellular OAH activity of the wild type strain they originate from as detected in a model reaction, preferably between 0.5 and 90, more preferably between 0.5 and 80, even more preferably between 1 and 50, most preferably between 1 and 25 and even most preferably between 1 and 10. According to another preferred embodiment, the oxalate deficient A. niger strains have an intracellular OAH activity, which is ranged between 0.1 and 100% of the intracellular OAH activity of the CBS 513.88 deposited strain as detected in a model reaction. More preferably, the oxalate deficient A. niger strains of the invention are strains having an intracellular OAH activity, which is ranged between 1 and 90% of the intracellular OAH activity of the CBS 513.88 deposited strain as detected in a model reaction.

The existence of such oxalate deficient strains still having a detectable OAH activity, is surprising, since it was thought that OAH was the only molecule responsible for the formation of oxalate. Mutants still having detectable level of OAH activity have several advantages compared to oxalate deficient strains with no detectable OAH activity (Pedersen H et al, Metabolic Eng. (2000) 2, 34-41): they are able to produce at least the amount of a given polypeptide the wild type strain would produce under the same culture conditions. Furthermore, the endogenous metabolic pathway of organic acids is most likely not pertubated.

According to a further preferred embodiment, the oxalate deficient A. niger strain of the invention is characterized by the fact that when this strain has been transformed with an expression construct comprising a gene coding for a polypeptide, said strain produces at least the amount of the polypeptide the wild type strain it originates from would produce under the same culture conditions, when the wild type strain has also been transformed with the same expression construct as the oxalate deficient strain.

The gene coding for the polypeptide to be produced may be homologous or heterologous to the oxalate deficient A. niger strain used. The term “heterologous” means that the polypeptide is not native to the A. niger cell. Preferably, the gene comprised in the expression construct is a heterologous gene for A. niger.

Preferred heterologous polypeptide is human serum albumine, lactoferrin, chymosin or Phospholipase A2. According to a preferred embodiment of the invention, the oxalate deficient strain has been transformed with a DNA construct comprising a DNA sequence encoding said polypeptide. Preferably, the polypeptide is an enzyme. Enzymes that can be produced are carbohydrases, e.g. cellulases such as endoglucanases, β-glucanases, cellobiohydrolases or β-glucosideases, hemicellulases or pectinolytic enzymes such as xylanases, xylosidases, mannanases, galactanases, galactosidase, rhamnogalacturonases, arabanases, galacturonases, lyases, or amylolytic enzymes; phosphatases such as phytases, esterases such as lipases, proteolytic enzymes, oxidoreductases such as oxidases, transferases, or isomerases. Preferably, the amylolytic enzyme to be produced is an alpha amylase (EC 3.2.1.1., alpha-1,4-glucan-4-glucano hydrolase or EC 3.2.1.2). More, preferably, the DNA sequence encodes a fungal alpha amylase. Most preferably, the DNA sequence encoding the fungal alpha amylase is derived from A. niger or Aspergillus oryzae. According to another embodiment, the enzyme to be produced is a proline specific endoprotease (EC 3.4.16.2). According to another embodiment, the enzyme to be produced is a phospholipase A1 (PLA1) (EC 3.1.1.32). More, preferably, the DNA sequence encodes a fungal PLA1. Most preferably, the DNA sequence encoding the fungal PLA1 is derived from Aspergillus niger or Aspergillus oryzae.

The DNA sequence encoding the polypeptide to be produced may be operably linked to appropriate DNA regulatory regions to ensure a high level of expression of said DNA sequence and preferably a high secretion level of said polypeptide. If the polypeptide to be produced is native to Aspergillus niger, its native secretion signal is preferably used. Alternatively, if the polypeptide to be produced is not native to Aspergillus niger, a fusion construct is preferably made comprising the glucoamylase gene of Aspergillus niger fused to the heterologous gene to be produced. According to a preferred embodiment of the invention, the regulatory regions of the Aspergillus oryzae alpha amylase gene are used. According to a more preferred embodiment of the invention, the regulatory regions of the A. niger glucoamylase gene are used. According to a preferred embodiment of the invention, the alpha amylase secretion signals are used. The DNA construct may also comprise a selectable marker. Alternatively, the selectable marker may be present on a second DNA construct. By way of example these markers include but are not limited to amdS (acetamidase genes), auxotrophic marker genes such as argB, trpC, or pyrG and antibiotic resistance genes providing resistance against e.g. phleomycin, hygromycin B or G418. Preferably, the marker gene is the acetamidase gene from Aspergillus nidulans. More preferably, the acetamidase gene from Aspergillus nidulans is fused; to the gpdA promoter. Transformation methods of A. niger are well-known to the skilled person (Biotechnology of Filamentous fungi: Technology and Products. (1992) Reed Publishing (USA); Chapter 6: Transformation pages 113 to 156). The skilled person will recognize that successful transformation of A. niger is not limited to the use of vectors, selection marker systems, promoters and transformation protocols specifically exemplified herein. After transformation, typically, the A. niger population is cultivated on a solid medium in a petri dish. The transformants selected after culture on solid medium are typically cultivated in flask during three to seven days to check for expression of the polypeptide.

Typically, for producing the polypeptide in the oxalate deficient A. niger strain in an industrial setting, a fed-batch fermentation process may be used. At the end of the fermentation, the polypeptide can be purified following techniques known to the skilled person. An example of such a recovery technique is explained in the following. When the fermentation is stopped, the host must be killed. This is accomplished by adding a killing-off agent at some specific temperature where this agent can work effectively. For example, the killing-off agent may be natriumbenzoate or kaliumsorbate. Depending on the identity of the killing-off agent chosen, the broth temperature is adjusted to the corresponding working temperature of this agent, by using classical cooling methods known to the skilled person. In the case of a polypeptide which is secreted into the fermentation medium, the separation of the cell material from the polypeptide is for example a simple filtration process: the fermentation broth is filtrated using a membrane filter press equipped with a textile cloth (membrane filter press and textile cloth can be obtained from Harborlite). To improve the filtration performance, a suitable filter-aid can be used, together with a suitable pre-coat of the filter cloth.

To remove any remaining small particles, additional filtration steps can be carried out, in such a way that a clear filtrate can be obtained. The filtrate can be polished filtered on filter plates with an average pore size of typically 1-10 micron. Several types of filter plates are known to the skilled person and are here suitable. Subsequently, a germ filtration may be carried out using a filter with a pore size of about 0.4 micrometer, to remove the major part of microorganisms. With these two filtrations, a pre-coat may be used to improve the filtration performance. The filtrate may be then concentrated by ultrafiltration (UF) with a factor of typically 10-25. Several types of UF membranes are suitable here. During UF molecules with a typical molecular weight of less than a few thousands (depending also on the shape of the molecules) are removed from the filtrate. Thus, the relative amount of low molecular weight molecules to the polypeptide of interest may be reduced about 10-25 times after UF. The duration of the UF varies depending on the viscosity and filterability of the filtrate (which varies due to natural variations in the raw materials). At that stage, the concentration of the polypeptide present in the ultrafiltrate is usually high enough to proceed with the formulation of the polypeptide into either a liquid or a dry formulation depending on the application contemplated.

A method was developed for obtaining oxalate deficient A. niger strains which are suitable for producing high yields of a polypeptide and which can be used as polypeptide producers in an industrial setting. The polypeptide may be homologous or heterologous for said A. niger. In case of a heterologous polypeptide or enzyme, the wild type strain on which the method of the invention is applied may have been earlier transformed to express a gene coding for such polypeptide or enzyme as has been described earlier in the description. Such oxalate deficient A. niger strains produce at least the amount of polypeptide the wild type strains they originate from produce under the same culture conditions. Preferably, the oxalate deficient A. niger strains produce more polypeptide than the wild type strain they originate from under the same culture conditions. According to another preferred embodiment, the mutants produce at least the amount of polypeptide the A. niger strain CBS 513.88 produced under the same culture condition. More preferably, the mutants produce more polypeptide than the A. niger strain CBS 513.88 produced under the same culture conditions.

This method comprises the following steps:
a) A. niger is subjected to UV irradiation,
b) MTP cultures of surviving colonies obtained in a) are realized
c) a selection within the MTP cultures is performed in which mutants are selected that produce no more than half the amount of oxalate that the wild type strain they originate from produces under the same culture conditions,
d) a second selection is performed within the mutants obtained in step c) in which mutants are selected that produce at least the amount of polypeptide the wild type strains they originate from produce under the same culture conditions.

According to a preferred embodiment, the method comprises the following steps:
a) culture conditions are developed, which allow a production of at least 15 mM oxalate in microtiterplates (MTP) or at least 30 mM oxalate in flask culture in the fermentation medium at the end of fermentation,
b) A. niger is subjected to UV irradiation,
c) MTP cultures of surviving colonies obtained in b) are realized under the culture conditions retained in a),
d) a selection within the MTP cultures is performed in which mutants are selected that produce no more than half the amount of oxalate that the wild type strain they originate from produces under the same culture conditions,
e) a second selection is performed within the mutants obtained in step d) in which mutants are selected that produce at least the amount of polypeptide the wild type strains they originate from produce under the same culture conditions.

According to another preferred embodiment, the method comprises the following steps:
a) culture conditions are developed, which allow a production of at least 15 mM oxalate in microtiterplates (MTP) or at least 30 mM oxalate in flask culture in the fermentation medium at the end of fermentation,
b) A. niger conidiospores are subjected to UV irradiation,
c) MTP cultures of surviving colonies obtained in b) are realized under the culture conditions retained in a),
d) a selection within the MTP cultures is performed in which mutants are selected that produce no more than half the amount of oxalate that the wild type strain they originate from produces under the same culture conditions,
e) a second selection is performed within the mutants obtained in step d) in which mutants are selected that produce at least the amount of polypeptide the wild type strains they originate from produce under the same culture conditions.

According to another preferred embodiment, the method comprises the following steps:
a) A. niger is subjected to UV irradiation,
b) MTP cultures of surviving colonies obtained in a) are realized,
c) a selection within the MTP cultures is performed in which mutants are selected that produce at least the amount of polypeptide the wild type strains they originate from produce under the same culture conditions.
d) a second selection is performed within the mutants obtained in c) in which mutants are selected that produce no more than half the amount of oxalate that the wild type strain they originate from produces under the same culture conditions,

Each step of these processes is characterized further below.

According to a preferred embodiment of the invention, in a first step, colonies of A. niger are first cultivated in a medium which allows a production of at least 30 mM oxalate in MTP or at least 100 mM oxalate in flask culture in the fermentation medium at the end of fermentation. The fermentation time should be at least 3 days. It is further a preferred embodiment of the method that the pH of this medium does not need to be manually corrected. The pH of the medium of this step is maintained between 3 and 7, preferably between 3.5 and 6.5, more preferably between 4 and 6. Most preferably the pH of this medium is maintained between pH 5 and 6. At such a pH value, the production of oxalate is known to be high. The pH of the medium is preferably buffered with a solution of 2-[N-Morpholino]ethanesulfonic acid (MES) whose concentration is ranged between 0, 1 and 1 M, more preferably between 0.15 and 0.55 M. Most preferably the MES concentration is 0.5 M. A nitrogen source is present in the medium of this step. Preferably the nitrogen source is a nitrogen source, which does not result in the acidification of the fermentation medium as a result of its uptake by the cell. More preferably, the nitrogen source of the medium of this step is urea. According to a preferred embodiment of the present invention, the medium used in this step is the flask defined medium 2 (FDM2) (see example 1). According to a preferred embodiment of the present invention, the A. niger strain used in this step is WT2 or the A. niger strain CBS 513.88 (see experimental information).

In a second step, A. niger is subjected to UV irradiation so that the survival percentage is ranged between 0.01% and 60%. Preferably, the survival percentage is ranged between 0.05% and 50%. More preferably, the survival percentage is 0.1%. It is well known to the skilled person that conidiospores is the preferred material to mutagenize A. niger by physical or chemical means. Mutants may however also be obtained from mycelium cells. The selection method described herein may be applied to select mutants obtained from either conidiospores or mycelium cells.

In a third step, MTP cultures of the surviving population obtained in a second step is performed during at least 3 days.

At the end of the MTP culture of the third step, mutants can be selected in a fourth step on basis of their oxalate production (oxalate selection step). Preferably mutants are selected that produce no more than one third of the amount of oxalate that the wild type strain they originate from produces under the same culture conditions. More preferably, mutants are selected that produce no more than one fifth of the amount of oxalate that the wild type strain they originate from produces under the same culture conditions.

An assay to quantify the oxalate present in the medium that may be used is described in the Examples. For practical reasons, the best mutants (the lowest oxalate producers) are retained for further characterization. Preferably 5 to 50 mutants are retained for further characterization. Typically, after 7 days in flask cultivation, it can be checked that these selected mutants produce far less oxalate than the wild type strain: in the case described in FIG. 7, less than 5 mM oxalate is found in the fermentation medium of the mutants compared to 40-45 mM for the wild type strain. After 7 days of fermentation, it can further be checked whether the medium is less acidified by the selected mutants than by the wild type strain. It can also be checked by measurement of the biomass produced and by measurement of the residual glucose concentration at different intervals during fermentation that the low level of oxalate measured in the mutants is not the consequence of either a poor growth and/or a poor metabolic activity of the selected mutants.

A second selection step which can be applied to the mutants before or after the oxalate selection step is the following: select mutants that produce at least the amount of polypeptide the wild type strains they originate from produce under the same culture conditions. Preferably, the mutants produce more of a given polypeptide than the wild type strains they originate from under the same culture conditions. According to another preferred embodiment, the mutants produce at least the amount of a given polypeptide the A. niger strain CBS 513.88 produced under the same culture condition. More preferably, the mutants produce more of a given polypeptide than the A. niger strain CBS 513.88 under the same culture conditions. To perform this last step, the mutants obtained in the previous step and a wild type control are cultivated in liquid medium for at least three days in a suitable medium. Preferably, the cultivation is performed during at least five days. At the end of the culture, the amount of the polypeptide produced may be determined using a system for detection of said polypeptide as defined earlier on in the application. Preferably, if the polypeptide produced is an enzyme, the amount of active enzyme produced is determined by measurement of its activity in a model reaction (see examples).

An optional sixth step may be further applied to select for oxalate deficient A. niger strains having an intracellular OAH activity which is detectable as detected in a model reaction. Preferably, the model reaction is the one described in experimental information in the Examples. More preferably, this step allows the selection of oxalate deficient A. niger strains having an intracellular OAH activity, which is ranged between 0.1 and 100% of the intracellular OAH activity of the wild type strain they originate from as detected in a model reaction, preferably between 0.5 and 90, more preferably between 0.5 and 80, even more preferably between 1 and 50, most preferably between 1 and 25 and even most preferably between 1 and 10. According to another preferred embodiment, the oxalate deficient A. niger strains have an intracellular OAH activity, which is ranged between 0.1 and 100% of the intracellular OAH activity of the CBS 513.88 deposited strain as detected in a model reaction. More preferably, the oxalate deficient A. niger strains of the invention are strains having an intracellular OAH activity, which is ranged between 1 and 90% of the intracellular OAH activity of the CBS 513.88 deposited strain as detected in a model reaction.

The invention also relates to the use of an oxalate deficient A. niger strain for producing a given polypeptide. Accordingly, the invention also relates to a method for producing a given polypeptide wherein an oxalate deficient A. niger as defined in this application is used. Such strain produces at least the same amount of said polypeptide as the wild type strain it originates from under the same culture conditions. Preferably, the strain produces more of said polypeptide than the wild type it originates from under the same culture conditions. According to another preferred embodiment, the strain produces at least the same amount of said polypeptide or enzyme as the CBS 513.88 A. niger strain under the same culture conditions. More preferably, the strain produces more of said polypeptide or enzyme than the CBS 513.88 A. niger strain under the same culture conditions.

### BRIEF DESCRIPTION OF THE DRAWINGS

FIG. 1 depicts the oxalate assay standard curve. The measured optical density is given as a function of the oxalate concentration present in solution.

FIG. 2 depicts the evolution of the pH of the culture supernatant of wild type A. niger during fermentation in FDM1 medium with or without pH correction.

FIG. 3 depicts the average oxalate production obtained during fermentation of the wild type A. niger in the FDM1 medium with or without pH correction.

FIG. 4 depicts the average oxalate production obtained during fermentation of the wild type A. niger in the FDM1 medium as a function of the MES concentration, with ammonium or urea as nitrogen source, without pH correction.

FIG. 5 depicts the average oxalate production obtained during fermentation of the wild type A. niger in the FDM2 medium without pH correction.

FIG. 6 depicts the pH evolution during fermentation of wild type and some selected oxalate deficient A. niger in the MDM1 medium.

FIG. 7 depicts the average alpha amylase produced after fermentation in the FDM2 medium by the wild type and the 34 mutants as a function of their oxalate production.

FIG. 8 depicts the measured OAH activity in three oxalate deficient A. niger mutants and in the wild type.

FIG. 9 depicts the average oxalate production obtained during the fermentation of the wild type and oxalate deficient A. niger in the FDM2 medium without pH correction.

FIG. 10 depicts the residual glucose concentration present during fermentation of wild type and oxalate deficient A. niger in the FDM 2 medium.

FIG. 11 depicts the pH evolution of culture supernatants of wild type and oxalate deficient A. niger fermented in the FDM2 medium.

FIG. 12 depicts the evolution of the biomass produced during fermentation of the wild type and oxalate deficient A. niger in the FDM2 medium.

FIG. 13 depicts the production of a proline specific endoprotease in WT1 and in FINAL (mutant 22) comprising the same estimated copy numbers of the gene coding for the proline specific endoprotease.

FIG. 14 depicts the production of phospholipase A1 in WT1 and in FINAL (mutant 22) in shake flask.

### EXAMPLES

Experimental Information

Strains

WT 1: A. niger strain is used as a control for the level of oxalate, the level of a given polypeptide and the level of intracellular OAH activity. This strain is deposited at the CBS Institute under the deposit number CBS 513.88.

WT 2: WT 1 strain comprising several copies of an expression cassette comprising the A. oryzae alpha-amylase gene integrated in the genome. This gene was already described elsewhere (Wirsel et al., (1989), Mol. Microbiol. 3:3-14). The original signal sequence coded by the A. oryzae alpha-amylase gene was replaced by the one of the glucoamylase gene from A. niger. WT 2 was constructed and selected by techniques known to persons skilled in the art and described in EP 635 574 A1 and in WO 98/46772.

OAH Activity Assay

Shake flask fermentations of different A. niger strains were performed as decribed hereafter. Cells were cultivated at 30° C., 170 rpm for three days in 100 ml of OAH cultivation medium in 500 ml shake flasks without a baffle. The OAH medium is defined in Table 1 below. Then, the pH was shifted to 8 by addition of Na₂CO₃ and cells were cultivated for an additional 15 to 18 hours. Mycelium was harvested by filtration, washed with 0.9% (w/v) NaCl, frozen in liquid nitrogen and stored at −80° C. Frozen cells were disrupted in a mortar under liquid nitrogen and then suspended in the following extraction buffer: 100 mM MOPS buffer pH 7.5 (MOPS=Morpholino propanesulfonic acid), 2 mM MnCl₂, 20 mM DTT, 5% sucrose. The suspension was centrifuged for 20 min. at 14,000 r.p.m. at 4° C. in an Eppendorf centrifuge 5417R. 925 μl of the assay buffer (assay buffer: 100 mM MOPS pH 7.5/2 mM Mn²⁺) was pre-heated at 25° C. 25 μl of a 40 mM oxaloacetic solution was added to this preheated mix. The oxaloacetic solution was prepared by dissolving 0.053 g of oxaloacetic acid in 10 ml of the assay buffer. 50 μl of the suspension obtained after centrifugation was added to the preheated mix. OAH activity was determined according to the method described by Pedersen et al, 2000, Mol. Gen. Genet. 263:281-286. Briefly, oxaloacetate is used as substrate. The enzyme activity was determined from the rate of decrease of the absorbance (delta A/min) at 255 nm during 3 minutes with a time interval of 20 seconds and the absorption coefficient of oxaloacetate. The assay was carried out at 25° C.

TABLE 1
OAH medium
Trace Metal Solution

| | | |
|---|---|---|
| ZnSO₄.7H₂O | 0.143 | g |
| CuSO₄.5H₂O | 0.025 | g |
| NiCl₂.6H₂O | 0.005 | g |
| FeSO₄.7H₂O | 0.138 | g |
| MnCl₂.4H₂O | 0.060 | g |
| Water | up to 10 | ml |

OAH medium, pH = 2.5 or 4.5,

| | | |
|---|---|---|
| Sucrose | 20 | g |
| KH₂PO₄ | 1.5 | g |
| MgSO₄.7H₂O | 1 | g |
| NaCl | 1 | g |
| CaCl₂.2H₂O | 0.1 | g |
| NaNO₃ | 15 | g |
| Trace Metal solution | 0.5 | ml |

(Adjust pH to 2.5 with HCl)

| | | |
|---|---|---|
| Water | up to 1 | liter |

Protein Assay

The protein content in the samples was determined according the Coomassie Plus Protein assay with Bovine Serum Albumin as a standard according to the manufacturer's instructions (Pierce, product number 23236).

In Example 1, alpha amylase is given as an example of enzyme that can be produced by an oxalate deficient A. niger strain at a level which is at least the same as the one produced by the parental strain the mutant originate from under the same culture conditions.

### Example 1

### Method to Make Oxalate deficient Aspergillus niger Mutants which are High Polypeptide Producers

Oxalate Deficient A. niger Mutants were Made Starting from WT2.

1. Growth Media

Cultures were performed at 34° C., in 96-wells microtiter plates (MTPs) or 300 ml flasks with one baffle in a rotary shaker at a shaking speed of 220 rpm.

Flask precultures were inoculated with 17 000 spores per ml. 100 ml cultures were inoculated with 10 ml of preculture.

TABLE 2
Flask preculture medium 1 (FPM1), pH 5.5
(all components are given in grams per liter)

| | |
|---|---|
| Corn steep liquor | 20 |
| (Roquette-Fréres, | |
| France) | |
| Glucose.1H₂O | 22 |

TABLE 3
Flask defined medium 1 (FDM1), pH 6
(all components are given in grams per liter)

| | |
|---|---|
| Glucose.1H₂O | 82.5 |
| Maldex 15 | 25 |
| (Boom Mepel, | |
| Netherlands) | |
| Citric acid | 2 |
| NaH₂PO₄.1H₂O | 4.5 |
| KH₂PO₄ | 9 |
| (NH₄)₂SO₄ | 15 |
| ZnCl₂ | 0.02 |
| MnSO₄.1H₂O | 0.1 |
| CuSO₄.5H₂O | 0.015 |
| CoCl₂.6H₂O | 0.015 |
| MgSO₄.7H₂O | 1 |
| CaCl₂.2H₂O | 0.1 |
| FeSO₄.7H₂O | 0.3 |
| MES* | 30 |

(*2-[N-Morpholino]ethanesulfonic acid)

Flask defined medium 2 (FDM2), pH 6: the FDM2 medium had the same composition as FDM1 except that 15 grams per liter urea are present instead of 15 grams per liter (NH₄)₂SO₄. This medium contained 100 grams per liter MES instead of 30 grams.

TABLE 4
Microtiter plate defined medium 1 (MDM1), pH 6
(all components are given in grams per liter)

| | |
|---|---|
| Glucose.1H₂O | 15 |
| Citric acid | 2 |
| NaH₂PO₄.1H₂O | 1.5 |
| KH₂PO₄ | 3 |
| Urea | 5 |
| ZnCl₂ | 0.02 |
| MnSO₄.1H₂O | 0.1 |
| CuSO₄.5H₂O | 0.015 |
| CoCl₂.6H₂O | 0.015 |
| MgSO₄.7H₂O | 1 |
| CaCl₂.2H₂O | 0.1 |
| FeSO₄.7H₂O | 0.3 |
| MES* | 30 |

(*2-[N-Morpholino]ethanesulfonic acid)

2. Assay for Oxalate Detection in A. niger Culture Supernatant

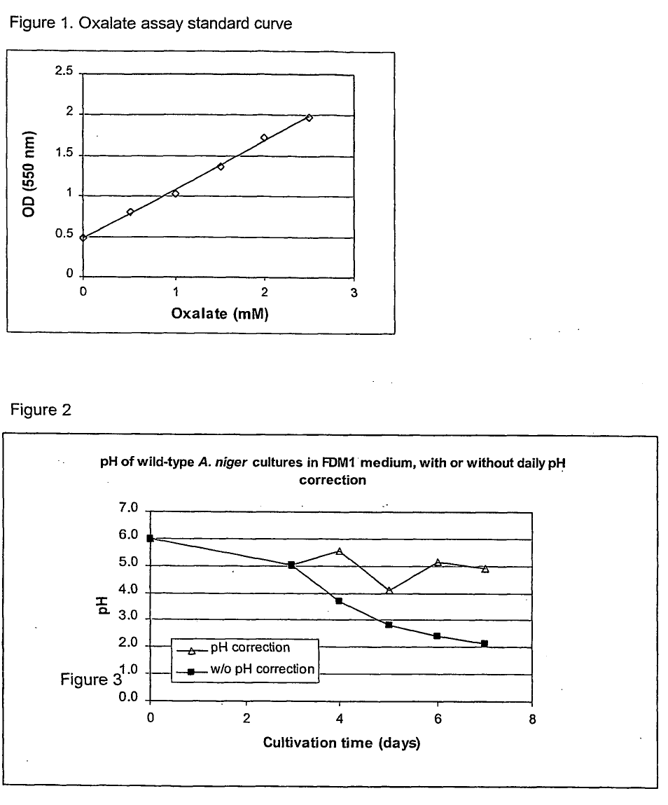

A commercial kit available from Sigma diagnostics (Sigma. OXALATE diagnostic kit, catalogus. nr. 591 year 2000-2001) was employed for oxalate quantification. The volumes recommended by the manufacturer were downscaled to reach a final assay volume of 48 μl, the assay being performed in 384-wells MTPs. A Beckman Multimek 96 was employed for all liquid transfers and the absorbance was read at 550 nm in a BMG spectrofluorimeter. The Oxalate assay standard curve is given in FIG. 1 (the optical density, OD, as a function of the oxalate concentration). In these conditions, the assay was found to be linear up to 2.5 mM.

3. Development of Cultivation Conditions to Maximize Oxalate Production

The wild-type strain employed throughout this section is WT 1.

The pH has been described as the most critical parameter for oxalate production. To achieve a high oxalate production, the pH of A. niger cultures should be maintained at a value close to 6 (Kubicek, C. P., et al, Appl. Environ. Microbiol. (1988) 54, 633-637; and Ruijter, G. J. G., et al,. Microbiology (1999) 145, 2569-2576). Oxalate production sharply decreases for pH values below 4 (Ruijter, G. J. G., van de Vondervoort, P. J. I., and Visser, J. 1999. Microbiology 145, 2569-2576). A pH close to 6 can hardly be maintained in A. niger cultures, because of the production of several organic acids by the fungus. To test how critical the pH of the culture was in the FDM1 medium, triplicate flasks cultures were performed with a wild-type A. niger strain, either with or without daily manual pH correction by addition of sterile sodium hydroxyde. A pre-culture phase of 48 hours in FPM1 medium was performed before FDM1 medium inoculation. In FDM1 medium, 0.15 M MES (30 g/L) was present to buffer the medium acidification during A. niger growth.

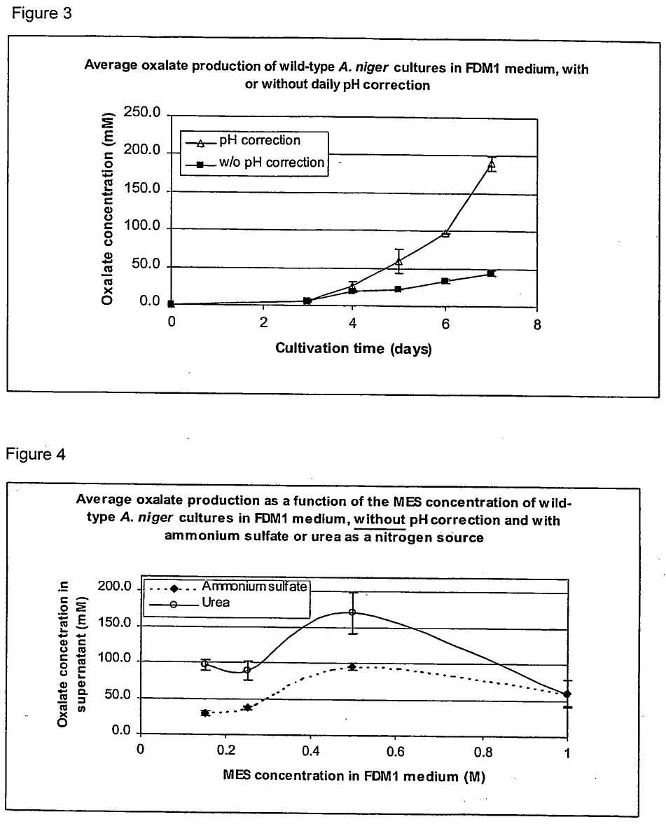

As can be seen in FIG. 2, the buffer present in the medium was not sufficient to counterbalance the production of organic acids by A. niger, and FIG. 3 shows that the oxalate yield was greatly affected by the pH of the culture. Cultures in which the pH was corrected yielded about 5 times more oxalate than the cultures in which the pH was not corrected.

During the screening for oxalate deficient strains, A. niger was grown in conditions yielding a maximal oxalate production, so that oxalate deficient strains could be selected and easily distinguished from a strain producing wild-type levels of oxalate. For practical reasons, a manual pH correction could not be an option to achieve a maximal oxalate production in the initial screening phase, when a huge number of mutants were still under evaluation. To improve the level of oxalate production without having the need to correct the pH of the cultures, two parameters were tuned in the FDM1 medium, which were the MES concentration in the medium and the nature of the nitrogen source.

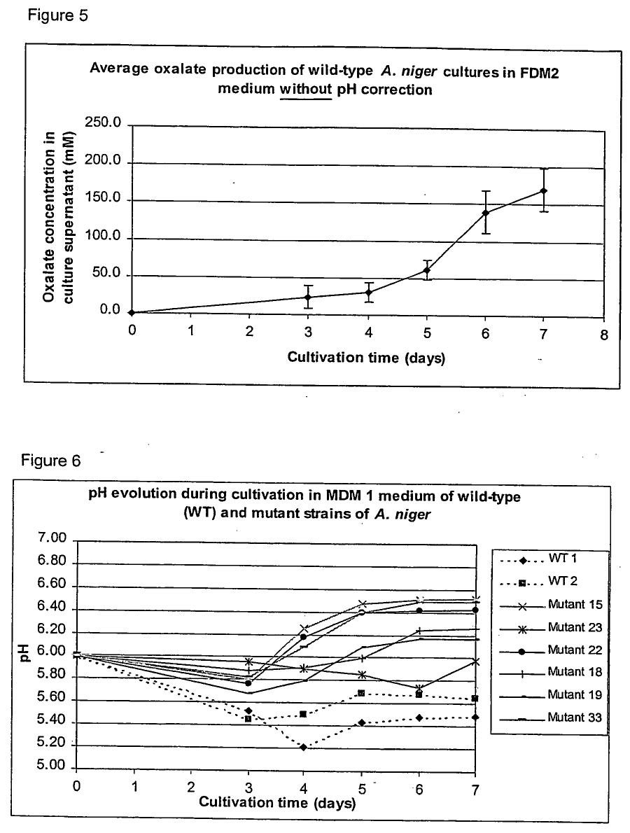

As shown in FIG. 4, increasing the MES concentration and replacing ammonium sulfate by urea had a major impact on the maximal oxalate concentration, which could be reached in A. niger cultures without pH correction. In FIG. 5, the maximal oxalate concentration reached after 6 or 7 days of fermentation depending on the composition of the fermentation medium is represented.

1M MES affected the growth of A. niger and an intermediate concentration of 0.5 M MES was chosen. Thus the growth medium finally chosen for flask cultivation during the screening was the FDM1 were the MES concentration was 0.5 M and where the ammonium sulfate was replaced by urea. From now on, that medium will be referred to as FDM2.

FIG. 5 shows that in FDM2, the oxalate concentration reached wthout pH correction was equivalent to the oxalate concentration reached in FDM1 with pH correction (compare with FIG. 3). So, there was no need for pH correction anymore.

4. First Selection: Oxalate Production

A. niger conidiospores were collected from WT 2 colonies sporulating on potato dextrose agar (PDA) medium (Difco, POTATO DEXTROSE AGAR, cultivation medium, catalogus. nr. 213400, year 1996-1997). 10 ml of a suspension containing 4×10⁶ conidiospores per ml was subjected to UV irradiation at 254 nm (Sylvania, 15 Watts Black Light Blue tube, model FT15T8/BLB) until an energy of 0.1783 J/cm² was received. A survival of 0.1% of the initial number of colonies was obtained. The mutagenized spores solution was plated on PDA medium and 10 000 survivors were picked using a Genomic Solutions Flexys colony picker and further grown into 96 wells microtiter plates (MTP). These MTPs, called “masterplates” were incubated at 34° C. until a strong sporulation was apparent.

The masterplates were replica plated using the Genomic Solutions Flexys colony picker into MTPs containing 40 μl of FPM1 and incubated for 48 hours at 34° C. 170 μl of MDM1 was then added and the MTPs were further incubated for 7 days at 34° C.

The supernatant of the 10 000 individual cultures was assayed for the presence of oxalate. In the cultivation conditions employed, the oxalate concentration reached in cultures of the WT 1 and WT 2 strains was in the range of 40 mM. The mutants for which the oxalate concentration in the growth medium was below 12 mM were selected for a further selection round. 255 mutants were retained. This second selection round was more stringent than the first one, so that it allowed to get rid of false positives.

The second mutant selection consisted of a quadruplicate MTP cultivation and assay for oxalate. The conditions employed were the same as the ones described here above. Table 5, second column below lists the oxalate concentration reached in the lowest producers amongst the mutants and in wild-type MTP cultures.

TABLE 5
Mutants

| | Average oxalate | Average alpha |
|---|---|---|
| | concentration | amylase activity |
| | (mM) | (U/ml) |
| 1 | 1.51 | 3.5 |
| 2 | 6.34 | 3.7 |
| 3 | 10.61 | 6.3 |
| 4 | 13.25 | 6.9 |
| 5 | 4.46 | 5.2 |
| 6 | 9.18 | 6.7 |
| 7 | 10.41 | 4.7 |
| 8 | 11.47 | 3.9 |
| 9 | 2.09 | 3.4 |
| 10 | 3.23 | 4.3 |
| 11 | 4.05 | 5.9 |
| 12 | 5.87 | 3.1 |
| 13 | 7.36 | 4.4 |
| 14 | 9.82 | 3.3 |
| 15 | 2.5 | 7 |
| 16 | 1.28 | 3.1 |
| 17 | 2.86 | 4.6 |
| 18 | 2.39 | 5.1 |
| 19 | 5.71 | 5.8 |
| 20 | 4.19 | 4.5 |
| 21 | 2.25 | 6 |
| 22 | 0.78 | 5.4 |
| 23 | 0.5 | 3.9 |
| 24 | 1.38 | 4.3 |
| 25 | 6.42 | 6.6 |
| 26 | 7.16 | 5.2 |
| 27 | 2.28 | 3.9 |
| 28 | 2.33 | 4.5 |
| 29 | 8.15 | 5.5 |
| 30 | 3.21 | 5.3 |
| 31 | 3.7 | 4.6 |
| 32 | 1.84 | 4.4 |
| 33 | 1.87 | 4.8 |
| 34 | 8.54 | 4.4 |
| WT 1 | 33.80 | — |
| WT 2 | 36.80 | 1.7 |

1 U/ml is the quantity of alpha amylase needed to convert 1 g soluble starch per hour into a product. The formation of this product is being measured by following the absorption at 620 nm after addition of iode at pH 5.5 and at 30° C. The incubation time with iode is between 15 and 25 minutes.

FIG. 6 shows that the selected mutant strains acidify less the MDM1 growth medium upon growth compared to the wild-type strains.

5. Second Selection: Alpha Amylase Production

As a second selection step, the 34 mutants obtained in the former paragraph were subsequently selected as to their capacities to produce alpha amylase.

The 34 mutants and WT2 were grown the same way as in the former paragraph, and characterized as to their alpha-amylase production.

The alpha-amylase activity present in culture supernatants was determined using the alpha amylase assay kit from Megazyme (Megazyme, CERALPHA alpha amylase assay kit, catalogus. ref. K-CERA, year 2000-2001). Table 5 third column lists the average alpha amylase production detected in WT2 and in the 34 mutants.

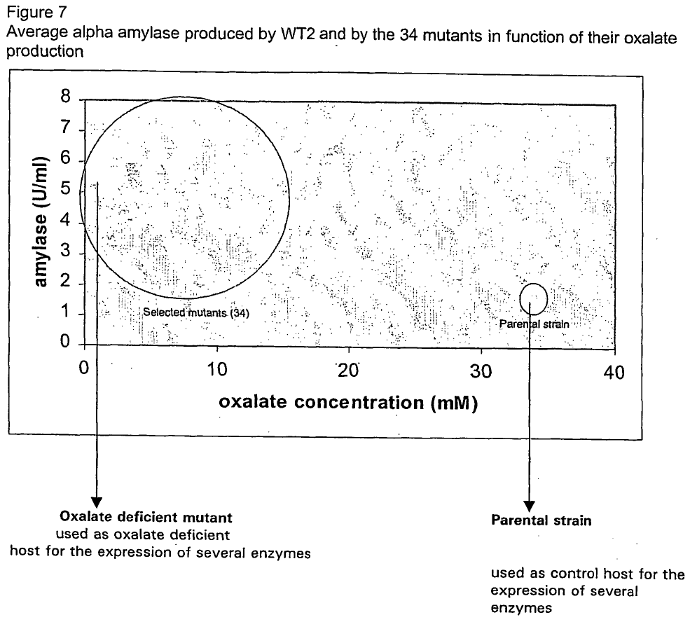

FIG. 7 depicts the average production of alpha amylase as a function of the oxalate production of the 34 mutants and the wild type. It could be observed in table 5, third column and in FIG. 7 that all the 34 mutants produced significantly more alpha-amylase than the wild-type strain they originated from. All the oxalate mutants found at the former paragraph were retained as mutants able to produce at least the same amount of enzyme as the wild type they originate from under the same culture conditions. Mutants 15, 19 and 22 were selected for further selection.

6. Third Selection: OAH Activity

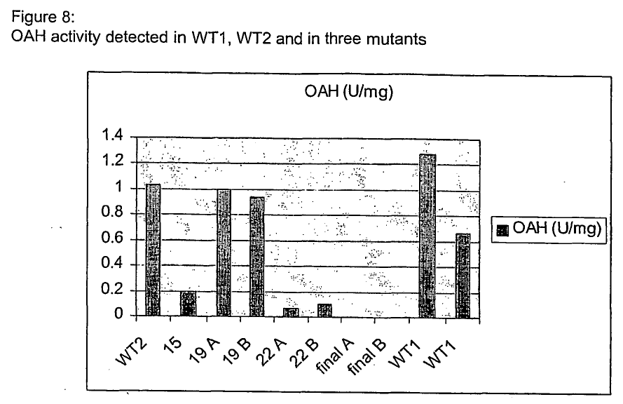

As an additional selection, the intracellular OAH activity was measured in the three mutants (15, 19, 22) selected at the former paragraph and as a control in WT1 and WT2. For some strains, measurements were made twice (A, B) as indicated in FIG. 8. The test developed to measure OAH activity is described in experimental data. Mutants 15 and 22 showed a detectable OAH activity (FIG. 8): approximatively 10 to 20% of the WT 1 or WT2. Surprisingly mutant 19 showed a high OAH activity, which is similar to the one of WT2. Surprisingly, these three oxalate deficient mutants still have a relative high OAH activity. Furthermore, they also have good enzyme production capacities.

### Example 2

### Characterisation of the A. niger Oxalate Deficient Mutants

1. Growth Media

TABLE 6
FPM1 and FDM2 media; as defined in example 1.
Flask preculture medium 2 (FPM2), pH 5.5
(all components are given in grams per liter)

| | |
|---|---|
| Maltose.1H₂O | 30 |
| Casein hydrolysate | 10 |
| Yeast extract | 5 |
| KH₂PO₄ | 1 |
| Tween 80 | 3 |
| MgSO₄.7H₂O | 0.5 |
| ZnCl₂ | 0.03 |
| CaCl₂ | 0.02 |
| MnSO₄ | 0.01 |
| FeSO₄.7H2O | 0.3 |

2. Characterization of the A. niger Oxalate Deficient Mutants

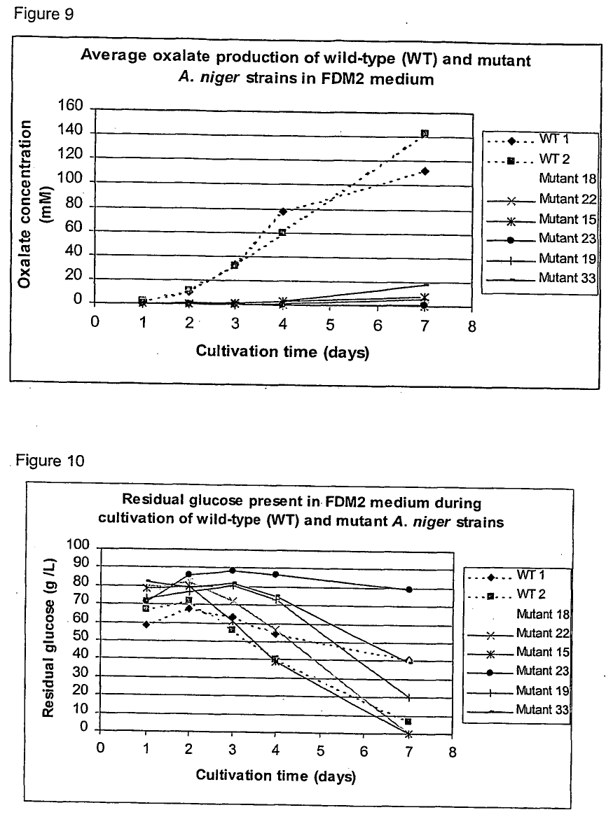

Mutants 18, 22, 15, 23, 19, 33 were grown in the FDM2 medium, after 48 hours of preculture phase in FPM1, and characterized as to their oxalate production, and several growth parameters (residual glucose, pH and biomass formed). The results obtained with the FDM2 medium confirmed the low level of oxalate production of the mutants compared to the wild-type strains (FIG. 9).

The residual glucose present in the FDM2 medium during growth of wild type and mutant strains was assayed using the Glucose assay kit from Sigma Diagnostics (Sigma, GLUCOSE diagnostic kit, catalogus nr. 510-A, year 2000-2001). As can be seen in FIG. 10, the glucose was almost completely consumed in some mutant cultures after 7 days of growth, suggesting the low oxalate level found in the selected mutant did not reflect a low metabolic activity. Only mutant 23 seemed to have a reduced metabolic activity.

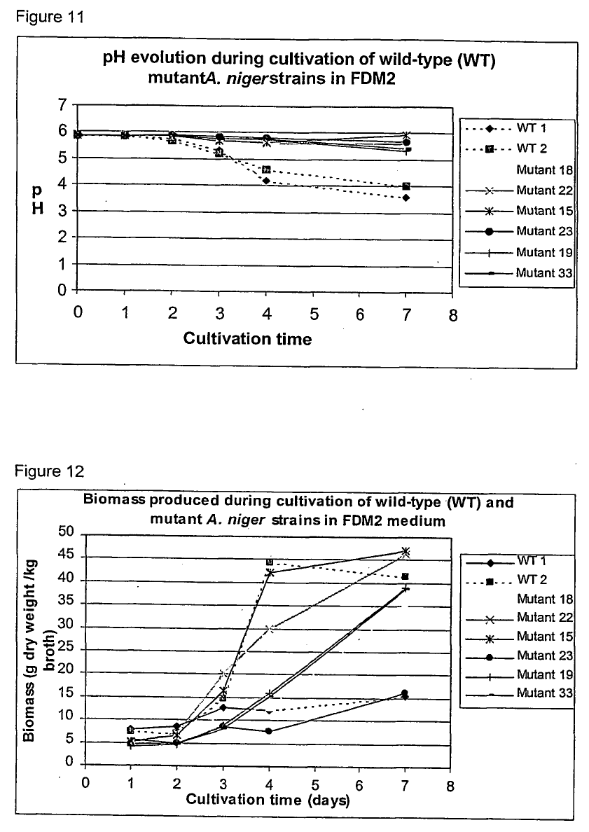

The pH of the cultures was also followed. As previously observed (see example 1), the acidification of the culture medium was less advanced in the mutants than in wild-type cultures (See FIG. 11).

Finally, to ensure that the reduced oxalate production of the mutants was not due to a poor growth, the biomass formation was followed by weighing the biomass dry weight formed in the cultures at various cultivation times. Flasks were sacrificed at each time interval considered and the total biomass dry weight content of the flask was determined.

As can be seen in FIG. 12, the mutants showed various growth profiles but tended to reach the same biomass level as the parental strain WT 2 after 7 days of cultivation. Mutant 23 was the only one which shown a low level of biomass formation, but this level was still comparable to the one reached by the wild-type strain WT 1 from which WT 2 originated. Mutant 23 was not retained as oxalate deficient mutant for further characterization. The sporulation capacities of the mutants were visually evaluated. It was found that the sporulation level of the mutants was comparable to the one of the wild type strain they originate from. Only one mutant seemed to have lower sporulation capacities.

In the following examples, mutant 22 was used as oxalate deficient A. niger strain for producing different enzymes. This mutant was obtained from WT2 and earlier on from WT1. In order to express other enzymes in this mutant, all the copies of the alpha amylase gene were deleted according to the method described in EP 635 574 A, using the acetamidase gene as selection marker gene. This mutant empty of any foreign enzyme encoding gene would be named FINAL in the following examples. Subsequently, FINAL was transformed with expression construct comprising the gene coding for the corresponding enzyme to be expressed as described in the following examples. In order to express specific enzymes in WT1, the expression constructs introduced in FINAL were also introduced in WT1 as described in the following examples. Copy number was checked. Mutant 22 was tested and compared to WT1 for the production of a proline specific endoprotease and PLA1. Mutant 22 produced the same amount of all enzymes tested as the WT1 it originates from under the same culture conditions or even more.

### Example 3

### Comparison of the Production of a Proline Specific Endoprotease in the WT1 and in FINAL Strains

The gene coding for the proline specific endoprotease, which has been used has already been published elsewhere (WO 02/45524). In order to express the proline specific endoprotease described in WO 02/45524 in WT1 and in FINAL, the construct depicted in WO 02/45524 (pGBFIN11-EPO) was introduced in these strains by cotransformation as described in WO 02/45524.

Transformants with similar estimated copy number were selected to perform shake flask experiments in 100 ml of the medium as described in EP 635 574 A1 at 34° C. and 170 rpm in an incubator shaker using a 500 ml baffeled shake flask. After four days of fermentation, samples were taken to determine the proline specific endoprotease activity. The proteolytic activity of the proline specific endoprotease was spectrophotometrically measured in time at pH 5 and about 37° C. using Z-Gly(cine)-Pro(line)-pNA as a substrate. 1 U proline specific endoprotease is defined as the amount of enzyme which converts 1 micromol Z-Gly(cine)-Pro(line)-pNA per min at pH 5 and at 37° C.

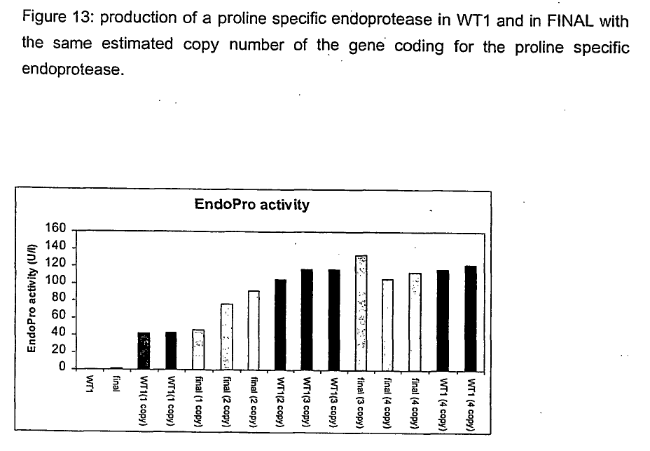

FIG. 13 shows that the proline specific endoprotease activity of the A. niger transformants with different estimated copy number is comprised in a range from 42 to 135 U/I. Strains with one estimated copy number have an activity of 42-46 U/I and correlates well with the activity of two and three copy strains. We concluded that FINAL produces at least the same amount of proline specific endoprotease as WT1 under the same culture conditions.

### Example 4

### Comparison of Phospholipase A1 (PLA1) Production in WT1 and in FINAL Strains

We chose to express PLA1 from A. oryzae in WT1 and in FINAL. The gene encoding this enzyme has already been published (Watanabe I, et al, Biosci. Biotechnol. Biochem. (1999), Vol 63, numero 5, pages 820-826). This gene was cloned into pGBFIN11 using the same technique as described in WO 02/045524 for the cloning of the proline specific endoprotease gene in pGBFIN11-EPO. This construct was introduced in these strains by cotransformation as described in WO 02/45524. Three independent transformants of WT1 and FINAL were tested for PLA1 expression in shakeflasks. The transformants with similar estimated copy number were cultivated in 100 ml of the same medium as described in EP 635 574 A1 at 34° C. and 170 rpm in an incubator shaker using a 500 ml baffeled shake flask. After 2, 3, 4, 5 days of fermentation, samples were taken to determine the PLA1 activity. To determine phospholipase PLA1 activity from Aspergillus niger (PLA1) spectrophotometrically, an artificial substrate is used: 1,2-dithiodioctanoyl phophatidylcholine (diC8, substrate). PLA1 hydrolyses the sulphide bond at the A1 position, dissociating thio-octanoïc acid. Thio-octanoic acid reacts with 4,4 dithiopyridine (color reagent, 4-DTDP), forming 4-thiopyridone. 4-Thiopyridone is in tautomeric equilibrium with 4-mercaptopyridine, which absorbs radiation having a wavelength of 334 nm. The extinction change at that wavelength is measured. One unit is the amount of enzyme that liberates of 1 nmol thio-octanoic acid from 1,2-dithiodioctanoyl phosphatidylcholine per minute at 37° C. and pH 4.0.

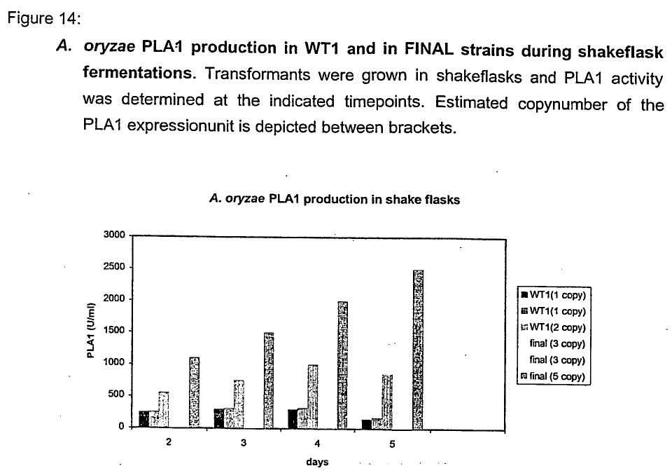

The substrate solution is prepared by dissolving 1 g diC8 crystals per 66 ml ethanol and add 264 ml acetate buffer. The acetate buffer comprises 0.1 M Acetate buffer pH 3.85 containing 0.2% Triton-X100. The colour reagent is a 11 mM 4,4-dithiodipyridine solution. It was prepared by weighting 5,0 mg 4,4-dithiodipyridine in a 2 ml eppendorf sample cup and dissolving in 1.00 ml ethanol. 1.00 ml of milli-Q water was added. The results are depicted in FIG. 14. It is shown that PLA1 activity in transformants of WT1 cultures decreased after 4-5 days. However, the PLA1 activity of transformants of FINAL accumulates during fermentation and no decrease in activity could be observed. We concluded that FINAL produces more PLA1 than the wild type counterpart it originates from under the same culture conditions.

1. An oxalate deficient A. niger strain for the production of a given enzyme, wherein the oxalate deficient strain produces at least the same amount of the enzyme as the wild type strain it originates from under the same culture conditions.

2. An oxalate deficient A. niger strain according to claim 1, wherein the oxalate deficient strain produces more of the enzyme than the wild type strain it originates from under the same culture conditions.

3. An oxalate deficient strain according to claim 1, wherein the oxalate deficient strain has an intracellular OAH activity, which is between 1% and 25% of the intracellular OAH activity of the wild type strain it originates from as detected in a model reaction.

4. An oxalate deficient A. niger strain, characterized in that when the strain has been transformed with an expression construct comprising a gene coding for an enzyme, said strain produces at least the amount of the enzyme the wild type strain it originates from would produce under the same culture conditions, when the wild type strain has been transformed with the same expression construct as the oxalate deficient strain.

5. An oxalate deficient A. niger strain according to claim 4, characterized in that the gene is an heterologous gene.

6. An oxalate deficient A. niger strain according to claim 1, wherein the strain produces at least the amount of enzyme the A. niger strain CBS 513.88 produced under the same culture condition, preferably more.

7. An oxalate deficient A. niger strain according to claim 1, wherein the enzyme is a fungal alpha amylase.

8. An oxalate deficient A. niger strain according to claim 7, wherein the fungal alpha amylase is derived from Aspergillus oryzae or A. niger.

9. A method for obtaining oxalate deficient A. niger strains which are suitable for producing at least the amount of enzyme the wild type strains they originate from produce under the same culture conditions, said method comprises the following steps:
a) A. niger is subjected to UV irradiation,
b) MTP cultures of surviving colonies obtained in a) are realized under the culture conditions retained in a),
c) a selection within the MTP cultures is performed in which mutants are selected that produce no more than half the amount of oxalate that the wild type strain they originate from produces under the same culture conditions,
d) a second selection is performed within the mutants obtained in step c) in which mutants are selected that produce at least the amount of enzyme the wild type strains they originate from produce under the same culture conditions.

10. A method according to claim 9, wherein the method comprises an additional step e) wherein mutant selected in step d) are further selected to have an intracellular OAH activity, which is between 1% and 25% of the intracellular OAH activity of the wild type strain it originates from as detected in a model reaction.

11. A method of producing a given enzyme comprising using an oxalate deficient A. niger strain according to claim 1.
